(19) United States
(12) Patent Application Publication
(10) Pub. No.: US 2008/0027288 A1
(43) Pub. Date: Jan. 31, 2008

(54) RFID enabled apparatus for monitoring a medical status and a method thereof

(76) Inventor: Jorg Renz, Lake Zurich, IL (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/496,366

(22) Filed: Jul. 31, 2006

(51) Int. Cl. A61B 5/00

(52) U.S. Cl. 600/300

(57) ABSTRACT

The invention provides a system and method for monitoring a medical status of a user. The system and method includes a radio frequency identification transmitter for transmitting the medical status of the user, a radio frequency identification receiver for receiving the medical status of the user and comparing the medical status of the user to a first threshold value and a second threshold value, and a network for communicating the received medical status of the user to a second user.

### BACKGROUND OF THE INVENTION

1. Field of the Invention

The present invention relates generally to radio frequency identification (RFID) for performing tasks. More specifically, the present invention relates to a method and system for retrieving patient information using RFID.

2. Description of the Related Art

Medical monitoring equipment is critical for the well being of a patient in a hospital or medical setting. The equipment monitors a patient's temperature, heart rate, blood pressure, respiratory functions, glucose level and the like. Currently, a patient is connected to the medical monitoring equipment via tubes and wires. When a patient has to perform bodily functions, the patient is either disconnected from the equipment by a health care professional or the patient uses portable medical monitoring equipment that is transported on wheels via a tray containing the medical monitoring equipment.

One of the problems of using a healthcare professional to disconnect the medical monitoring equipment is that the healthcare professional is also needed to reconnect the medical monitoring equipment. Errors can occur in reconnecting the wires and tubes. Also, the wear and tear on the wires and tubes increases over time from connecting and disconnecting the wires and tubes.

Another problem with using a healthcare professional to connect and disconnect wires and tubes is that it is an inefficient use of human resources. With healthcare costs increasing and more responsibilities being given to healthcare professionals, it makes business sense to utilize healthcare professionals such that they are used in a manner that maximizes profits and provides superior customer service.

Some medical facilities keep the patient immobile and utilize a bed pan. However, patients sometimes feel uncomfortable and embarrassed using a bed pan. Since a patient is in an unfamiliar environment, the additional stress of having to use a bed pan can affect readings on the medical monitoring equipment.

Conventional portable medical monitoring equipment is inconvenient because the patient has to roll the equipment around. When the patient has to use the bathroom, the portable medical equipment has to be positioned in the proper manner. Also, maneuverability of the patient is affected because the patient has to deal with opening a door as well as maintaining control of the monitoring medical equipment. For example, a patient may only need two functions monitored. However, the medical monitoring equipment may be an all purpose machine that has a plurality of functions. This results in a bulky piece of equipment.

Typically, if there is an alarm where the medical monitoring equipment detects a situation where a threshold has been exceeded, a nurse arrives to investigate the situation. If the situation is beyond the expertise of the nurse, a doctor is paged. Crucial time between the alarm and the summoning of the doctor can be wasted in this situation. It is also possible that the wrong type of doctor can be summoned.

A further problem with conventional medical equipment is that remote diagnostic is inconvenient for the patient. For example, a patient typically has to connect to bulky medical equipment and either call in to a physician after reading the data from the medical equipment or connect the medical equipment to a phone line after taking a reading. This affects a person's schedule because now the patient has to take time out of their schedule to perform these tasks.

Thus, there is a need for medical monitoring equipment that is portable, does not significantly affect the manner in which a patient performs every day functions, provides an alarm indication to a remote user, and is modular.

### SUMMARY OF THE INVENTION

The above mentioned problems in the prior art can be substantially accomplished by a system and method for monitoring a medical status of a user.

According to an aspect of the invention for realizing the above objects, there is provided a system and method including a radio frequency identification transmitter for transmitting the medical status of the user, a radio frequency identification receiver for receiving the medical status of the user and comparing the medical status of the user to a first threshold value and a second threshold value, and a network for communicating the received medical status of the user to a second user.

According to another aspect of the present invention, there is provided a system for monitoring a medical status of a patient. The system comprises a radio frequency identification transmitter for transmitting the medical status of the user, a radio frequency identification receiver for receiving the medical status of the user and selecting a contact person based on at least one of a comparison of the medical status of the user to a first threshold value and a second threshold value and a medical condition being monitored, and a network for communicating a history of the received medical status of the user to a second user.

According to a further aspect of the present invention, there is provided a system for monitoring a medical status of a patient wherein the radio frequency identification transmitter comprises at least one of a thermometer, cholesterol monitor, glucose monitor, heart rate monitor, respiratory monitor and a blood pressure monitor.

According to still another aspect of the present invention, there is provided a system for monitoring a medical status of a patient. The system comprises a radio frequency identification transmitter for transmitting the medical status of the user, a radio frequency identification receiver for receiving the medical status of the user and selecting a contact person based on at least one of a predetermined time selected by at least one of a doctor or the patient, and a network for communicating a history of the received medical status of the user to the doctor.

### BRIEF DESCRIPTION OF THE DRAWINGS

Exemplary embodiments of the present invention will be set forth in detail with reference to the drawings, in which like reference numerals refer to like elements:

FIG. 1 is a diagram illustrating a radio frequency identification (RFID) monitoring system in accordance with an embodiment of the present invention;

FIG. 2 is a flowchart illustrating a process for monitoring a medical status in accordance with an embodiment of the present invention; and

FIG. 3 is a diagram illustrating an exemplary data processing system in accordance with an embodiment of the present invention.

### DETAILED DESCRIPTION OF EXEMPLARY EMBODIMENTS

Several exemplary embodiments of the present invention will now be described in detail with reference to the accompanying drawings. In the drawings, the same or similar elements are denoted by the same reference numerals even though they are depicted in different drawings. In the following description, a detailed description of known functions and configurations incorporated herein has been omitted for conciseness.

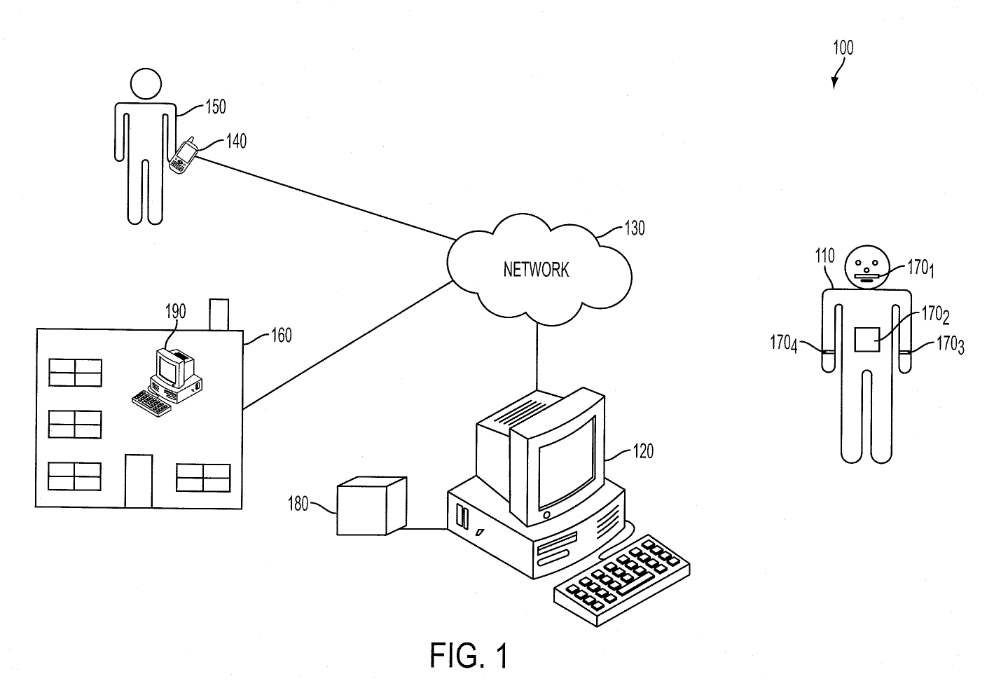

FIG. 1 is a diagram illustrating a radio frequency identification (RFID) monitoring system 100 in accordance with an embodiment of the present invention. The monitoring system 100 comprises a first computer 120 or the like, a network 130, a communication devices 140, a second computer 190, an RFID medical status device transmitter 170 comprising 170₁, 170₂, 170₃ and 170₄ and an RFID medical status device receiver 180.

It should be appreciated by those skilled in the art that the network 130 may comprise a public switched network, a wireless network, a private packet network and/or the Internet. Similarly, it should be appreciated by those skilled in the art that communication device 140 may comprise a cellular phone, a pocket PC, a personal digital assistant (PDA), and/or a computer.

In one embodiment of the present invention, medical status device transmitter 170 may comprise an integrated unit wherein the RFID transmitter and the medical status device are integrated as one unit. In another embodiment of the present invention the RFID transmitter and the medical status device are separate units. In still another embodiment of the present invention, the RFID medical status device transmitter 170 comprises an all purpose device that can perform multiple functions or at least two functions such as pulse monitoring, cholesterol monitoring, glucose level monitoring, respiratory monitoring, temperature monitoring and the like. In another embodiment of the present invention, the RFID medical status device transmitter 170 comprises a single purpose device wherein each device performs a single function such as pulse monitoring, cholesterol monitoring, glucose level monitoring, respiratory monitoring, temperature monitoring and the like. In a further embodiment of the present invention, the RFID medical status device transmitter 170 comprising a single purpose device is modular wherein different RFID medical status device transmitter 170 can be connected to, for example, share a power source or a transmission channel.

A patient 110 is fitted with the RFID medical status device transmitter 170 either an all purpose model or a single function model. The application used may be dependent on the patient's medical condition, the costs involved and the purpose of the monitoring. In the embodiment of the invention shown in FIG. 1 multiple single purpose RFID medical status device transmitters 170 are shown for illustrative purposes. The RFID medical status device transmitter 170 may transmit continuously or periodically based on the type power supply, the transponder or receiver used.

The patient or a third party such as a medical professional can program the system 100 to select a contact person via the computer 120. In accordance with an embodiment of the present invention, the contact person can be a specialist related to the patient's 110 medical condition or status that is being monitored. The contact person can also be the patient's 110 personal physician. For instance, rather than have the patient 110 come to the doctor's office for testing, the personal physician can retrieve the patient's medical status remotely. This saves the patient the inconvenience of going in to the doctor's office where the patient's stress related to being in a doctor's office can affect the readings. In addition, the doctor can retrieve a report for a predetermined period of time covering a long period of time rather than the readings of a short duration that are obtained when a patient goes to the doctor's office.

In an embodiment of the present invention, RFID medical status device receiver 180 can be integrated into a portable communication device such as a PDA, pocket PC and the like without a network connection. In this embodiment, the location of the patient can be local to the position of the contact person wherein the location can comprise a home, office, school, supermarket, mall and the like. The contact person can be a nonmedical person such as a parent, spouse, coworker, teacher or any person capable of performing a monitoring function. For example, a parent may want to monitor a child's medical status while in the house with the child. The parent also has the option of transporting the child to any location and being in direct contact with the medical status of the child.

A minimum and maximum threshold can also be set for each medical device or feature of the all in one RFID medical status device transmitter 170 or for the single function RFID medical status device transmitter 170.

The measurement period can be selected for predetermined periods. For example, cholesterol testing and glucose testing can be set to be read six hours after the patient's 110 last meal so that the readings are reliable and not affected by the patient's recent food intake. The accuracy of the RFID medical status device transmitter 170 is preferably within ±0.5%. The operational frequency of the RFID medical status device transmitter 170 is preferably at least one of 433 MHz, 13.56 MHz and 2.45 GHz.

The RFID medical status device receiver 180 receives the signals from the RFID medical status device transmitter 170 and provides the information to the computer 120. It should be appreciated by those skilled in the art that RFID medical status device receiver 180 can be integrated into computer 120.

Computer 120 processes the signal and based on a program such as Solarian™ manufactured by Siemens Medical Solutions a determination can be made to contact a physician based on the predetermined upper threshold values being exceeded or the lower threshold values being unmet and the type and/or feature of the RFID medical status device transmitter 170 used such as cholesterol monitoring, glucose monitoring, blood pressure and the like.

A message comprising the medical status information and contact information is communicated over the network 130. Network 130 may comprise a private network or a public network such as a local area network (LAN), a wide area network (WAN), a public switched network (PSTN), an Internet, a packet network, a wireless network and the like.

The message can be received at a medical facility 160 such as a hospital or doctor's office via a second computer 190. It should be appreciated by those skilled in the art that the second computer 160 may comprise any type of communication device.

In an embodiment of the invention, the contact person 150 may be reached directly via the communication device 140 and provided with the medical status information. The contact person can then review the medical status information which in an embodiment of the invention covers a predetermined period. In another embodiment, Solarian can provide a customized report for the contact person wherein a correlation among all the medical status information such as cholesterol readings, glucose readings, temperature, blood pressure, pulse rate, respiratory rate and the like may be provided.

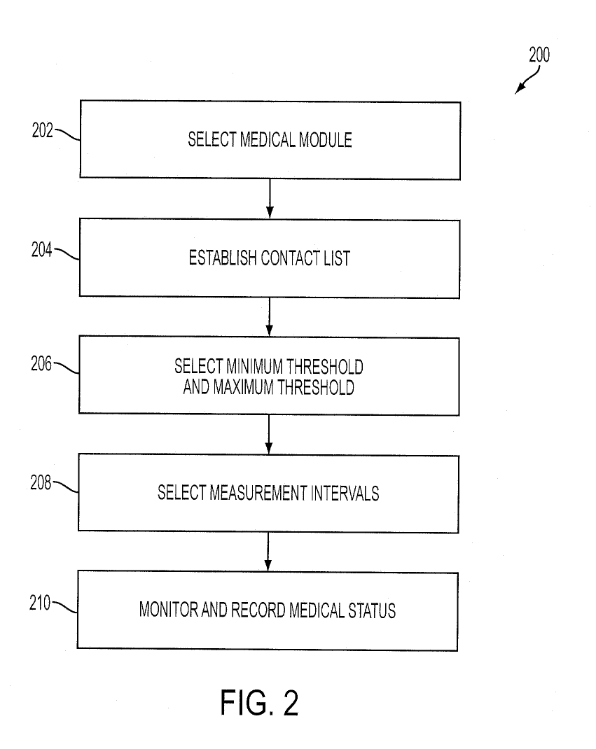

FIG. 2 is a flowchart illustrating a process 200 for monitoring a medical status in accordance with an embodiment of the present invention. The process 200 begins at step 202 where a medical module is selected. The medical module may comprise a thermometer, heart rate monitor, respiratory monitor and the like. A user may select one module or a plurality of modules. In accordance with an embodiment of the present invention, the modules can work independently from each other. In another embodiment of the present invention, the modules can work in combination with each other.

At step 204, a contact list is established. The contact list may be based on a personal choice of the patient or may have a relationship with the module used. For example, if a patient has a heart problem, selection of the heart rate monitor may automatically select a heart specialist. In another embodiment of the present invention, a database can be established where doctors are selected based on specialty and availability. Thus, if a patient is at a hospital, only doctors that are working onsite are contacted. In the case of a patient at home, the patient may have the option of selecting a personal doctor and have the doctor meet the patient at the hospital or select a doctor located at the nearest hospital or a hospital with an excellent reputation in a particular area.

At step 206, a minimum threshold and a maximum threshold are established are established for the selected medical module. The minimum threshold and the maximum threshold may also be reset to new values while the selected module is in use.

At step 208, measurement intervals are selected for each of the selected modules. The measurement intervals may be any portion or multiple of a second. For example, the transmitter 170 can transmit medical status information via RFID every five seconds to the receiver.

At step 210, the medical status information is monitored and recorded. A report can be prepared based on the received medical status. In addition, the medical status information can be combined with a patient's medical file. If there is an alarm due to a threshold being exceeded or fallen below, computer 120 can transmit the patient's medical status information and/or medical history over the network 130 to a doctor on the contact list. The medical status information can comprise a visual or verbal message. For example, a doctor on the contact list can be contacted via cell phone or landline phone and a verbal message provided comprising the medical status information of the patient. In another embodiment of the present invention, the doctor on the contact list can be contacted via the doctor's communication device 140 such as a cell phone, computer, and/or PDA and the medical status information of the patient provided via email.

It is to be understood that the present invention can be implemented in various forms of hardware, software, firmware, special purpose processes, or a combination thereof. In one embodiment, the present invention can be implemented in software as an application program tangible embodied on a computer readable program storage device. The application program can be uploaded to, and executed by, a machine comprising any suitable architecture.

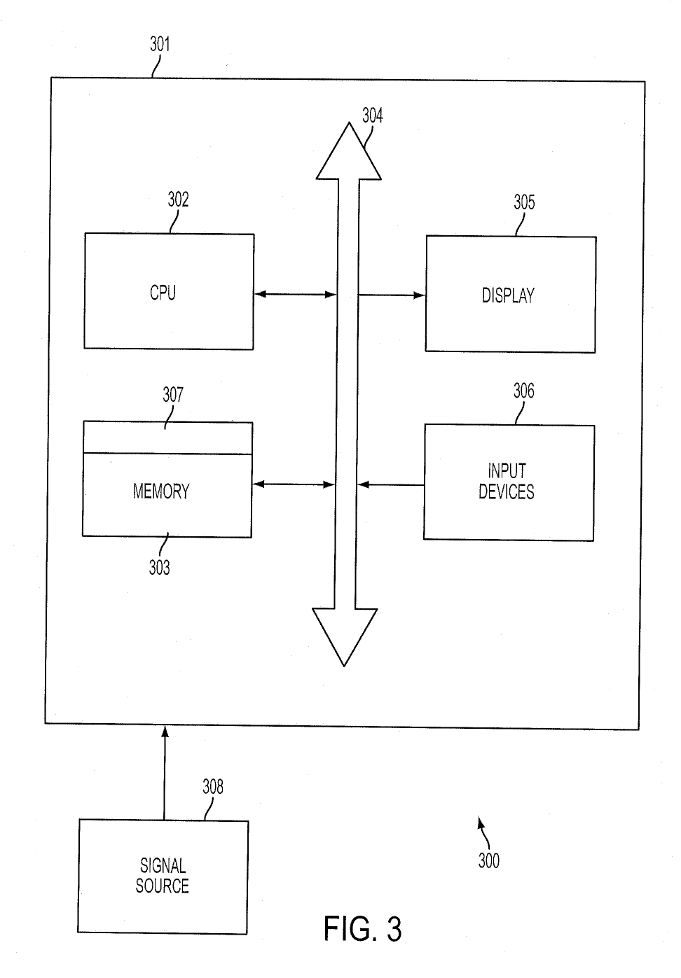

Referring now to FIG. 3, according to an embodiment of the present invention, a computer system 301 for implementing the present invention can comprise, inter alia, a central processing unit (CPU) 302, a memory 303 and an input/output (I/O) interface 304. The computer system 301 is generally coupled through the I/O interface 304 to a display 305 and various input devices 306 such as a mouse and a keyboard. The support circuits can include circuits such as cache, power supplies, clock circuits, and a communication bus. The memory 303 can include random access memory (RAM), read only memory (ROM), disk drive, tape drive, etc., or a combinations thereof. The present invention can be implemented as a routine 307 that is stored in memory 303 and executed by the CPU 302 to process the signal from the signal source 308. As such, the computer system 301 is a general purpose computer system that becomes a specific purpose computer system when executing the routine 307 of the present invention.

The computer system 301 also includes an operating system and micro instruction code. The various processes and functions described herein can either be part of the micro instruction code or part of the application program (or combination thereof) which is executed via the operating system. In addition, various other peripheral devices can be connected to the computer platform such as an additional data storage device and a printing device.

It is to be further understood that, because some of the constituent system components and method steps depicted in the accompanying figures can be implemented in software, the actual connections between the systems components (or the process steps) may differ depending upon the manner in which the present invention is programmed. Given the teachings of the present invention provided herein, one of ordinary skill in the related art will be able to contemplate these and similar implementations or configurations of the present invention.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed is:

1. A system for monitoring a medical status of a user comprising:
a radio frequency identification transmitter for transmitting the medical status of the user;
a radio frequency identification receiver for receiving the medical status of the user and comparing the medical status of the user to a first threshold value and a second threshold value; and
a network for communicating the received medical status of the user to a second user.

2. The system according to claim 1, wherein the medical status comprises a temperature.

3. The system according to claim 1, wherein the medical status comprises a blood pressure.

4. The system according to claim 1, wherein the medical status comprises a respiratory rate.

5. The system according to claim 1, wherein the network comprises a wireless network.

6. The system according to claim 1, wherein the network comprises a public switched telephone network.

7. The system according to claim 1, wherein the network comprises a private network.

8. The system according to claim 7, wherein the private network comprises a packet network.

9. The system according to claim 1, further comprising:
providing the medical status to at least one of a mobile phone, a personal digital assistant, a computer and a pager.

10. The system according to claim 1, further comprising:
continuously transmitting the medical status between the transmitter and receiver.

11. The system according to claim 1, further comprising:
selectively transmitting the medical status between the transmitter and receiver.

12. A method for monitoring a medical status of a user comprising:
transmitting the medical status of the user;
receiving the medical status of the user and comparing the medical status of the user to a first threshold value and a second threshold value; and
communicating the received medical status of the user to a second user.

13. The method according to claim 12, further comprising:
selectively transmitting the medical status.

14. The method according to claim 12, further comprising:
providing the medical status to at least one of a mobile phone, a personal digital assistant, a computer and a pager.

15. The method according to claim 12, wherein the step of transmitting comprises:
providing a temperature of the user.

16. The method according to claim 12, wherein the step of transmitting comprises:
providing a heart rate of the user.

17. The method according to claim 12, wherein the step of transmitting comprises:
providing a blood pressure of the user.

18. The method according to claim 12, wherein the step of transmitting comprises:
providing a respiratory rate of the user.

19. A system for monitoring a medical status of a user, the system comprising:
a radio frequency identification transmitter for transmitting the medical status of the user;
a radio frequency identification receiver for receiving the medical status of the user and
selecting a contact person based on at least one of a comparison of the medical status of the user to a first threshold value and a second threshold value and a medical condition being monitored; and
a network for communicating a history of the received medical status of the user to a second user.

20. The system of claim 19, wherein the radio frequency identification transmitter comprises at least one of a thermometer, cholesterol monitor, glucose monitor, heart rate monitor, respiratory monitor and a blood pressure monitor.

21. The system of claim 19, wherein the radio frequency identification transmitter comprises an integrated unit.

22. The system of claim 19, wherein the radio frequency identification transmitter comprises a modular unit.

23. The system of claim 19, wherein the medical status of the patient is communicated at predetermined intervals between the radio frequency identification transmitter and the radio frequency identification receiver.

24. The system of claim 19, wherein an operational frequency of the radio frequency identification transmitter and the radio frequency identification receiver comprises at least one of 433 MHz, 13.56 MHz and 2.45 GHz.

25. A system for monitoring a medical status of a user, the system comprising:
a radio frequency identification transmitter for transmitting the medical status of the user;
a radio frequency identification receiver for receiving the medical status of the user and
selecting a contact person based on at least one of a predetermined time selected by at least one of a doctor or the user; and
a network for communicating a history of the received medical status of the user to the doctor.

26. A system for monitoring a medical status of a patient, the system comprising:
a radio frequency identification transmitter for transmitting the medical status of the patient; and
a radio frequency identification receiver for receiving the medical status of the patient and providing the medical status of the patient to a user.
